(19) United States
(12) Patent Application Publication
(10) Pub. No.: US 2010/0035327 A1
(43) Pub. Date: Feb. 11, 2010

(54) Use of rice-derived products in a universal cell culture medium

(76) Inventors: Ann Marie Steele, Seminole, FL (US); Mark Dargan Smith, Santa Fe, NM (US); Somchai Boonchuen, Bangkok (TH)

Correspondence Address: Mark Dargan Smith, P.O. Box 4069, Santa Fe, NM, 87502, US

(21) Appl. No.: 12/228,172

(22) Filed: Aug. 11, 2008

(51) Int. Cl. C12N 7/00; C12N 5/02; C12N 1/20; C12N 1/14

(52) U.S. Cl. 435/235.1; 435/366; 435/404; 435/431; 435/252.1; 435/254.1; 435/349; 435/354; 435/353; 435/363

(57) ABSTRACT

The invention is used as an additive to a culture medium to maintain and grow various cells including stem cells and support their manufactured and secreted products including cytokines, chemokines and growth factors in an environment which provides:

a) metabolic enabling or enhancement of measurable parameters within the cellular population
b) availability and usage of nutrients to cells
c) distinct and positive effect on cellular dynamics (i.e. cellular proliferation and secretion of manufactured products by the cells)
d) stabilized environment for maintaining conditions for growth and other cellular and metabolic processes including secretion of manufactured products including signaling factors
e) enhanced biological cellular function of inherent cellular processes
f) non-interference with normal cellular metabolics (i.e. signaling)
g) increased protection from toxic effects to the cell
h) additional benefits to the cellular population which in absence of the additive would be lacking.

### BACKGROUND

1. Field of the Invention

The invention relates to the field of laboratory research, stem cell therapy, stem cell derived-product therapy, biotechnology and research, medical science, and molecular medicine.

2. Background of the Invention

There are no known drugs available for the treatment of progressive disorders of the central nervous system or neurodegenerative diseases such as Parkinson's disease, Huntington's disease and Alzheimer's disease. Likewise, there are no effective therapeutic methods for complete treatment and resolution of genetic and/or acquired diseases which precipitate or accompany injury, denaturation and heteroneoformation (fibrosis and degeneration) of tissues, such as cerebral infarction, obstructive vascular disease, myocardial infarction, cardiac failure, chronic obstructive lung disease, pulmonary emphysema, bronchitis, interstitial pulmonary disease, asthma, hepatitis B, hepatitis C, alcoholic hepatitis, hepatic cirrhosis, hepatic insufficiency, pancreatitis, diabetes mellitus, Crohn disease, inflammatory colitis, IgA glomerulonephritis, glomerulonephritis, renal insufficiency, decubitus, burn, sutural wound, laceration, incised wound, bite wound, dermatitis, cicatricial keloid, keloid, diabetic ulcer, arterial ulcer and venous ulcer.

Human cell biology anticipates a transformation generated by the recently acquired ability to derive, support and differentiate specific cell types and, by default, their manufactured products for unique biomedical and medical application; this ability will enable integration and translation of newly-identified biological potential into revolutionary therapeutic paradigms which have the potential to alter biomedicine and the clinical practice of medicine. Recent progress in stem cell biology has focused on deciphering and reproducing micro-environmental conditions consistent with induced and guided differentiation of human tissues and cells from cultured stem cells for the purpose of treating the diseases. Although all stem cells possess self-renewal capabilities, all are not pluripotent, but may be multipotent or unipotent and thus differentiation lineages may be various or restricted. Stem cells can be classified roughly into 5 types based on derivation: embryonic (ES cell), fetal, adult, cord blood and placental. The term “adult” used in conjunction to the present invention means a non-embryonic, non-fetal, somatic-derived/tissue-based stem/progenitor cells.

Since the ES (embryonic stem cell) cell and the early EG cell (embryonic germ cell) have pluripotency and are thought capable of giving rise to various progenitor lineages capable of differentiation into numerous adult cell types, they are drawing attention as the most potent cell source for use in tissue reconstruction. Fetal-derived cells (e.g. nerve stem cells) have been isolated from fetuses and cultured. Stem cell procurement from either embryo or fetus is injury-based and therefore presents an ethical problem. The properties of embryonic and adult stem/progenitors are believed to be based in part on methylation/demethylation events which impose functional restrictions inherent to embryonic vs. adult stem cells. In fact, the transplantation of embryonic stem cells into adult tissues have the very real propensity for tumor formation as the cells are not under appropriate regulatory control within an appropriate microenvironment. Although great strides are being made in our understanding of embryonic stem cell behavior and usage, it is realistically anticipated that their clinical usage is at least ten years away. This underscores the critical importance for the immediate and realistic use of biologically potent tissue-based stem cells and their manufactured products for current clinical applications!

In adult organs, the harvesting of the allogeneic/donor somatic tissues for derivation of stem/progenitors for purpose of transplantation carries risk of immune rejection sequelae. The long-term therapeutic safety of embryonic or fetal cells is difficult to establish given current biological knowledge. By contrast, use of autologous stem/progenitors and their manufactured products will enable, development of immediate treatment modalities, assessment of treatment efficacy and will do so without fear of rejection.

Being as stem cells possess an inherent ability for self-renewal under appropriate growing conditions, it is possible to produce these cells on a large scale using such in vitro growth environments. It is further possible, and necessary, to determine that stem cells expanded in culture and their derived products have the same/similar biological properties in vitro as in vivo. Given the preceding, these cells have the potential to provide a viable source of therapeutic cells in appropriate numbers for reparative transplantation or alternatively, can be “farmed” to derive unique stem-cell manufactured secreted products inherent to their own unique metabolism that are compatible with promotion of stem cell product-based healing modalities.

Tissue-based stem/progenitor cells have widely divergent proliferative abilities. In organs where stem cells appear to be rare, reparative attempts are often inadequate. Tissue specific stem/progenitors, although often unipotent, can be used in repair of a specific organ if delivered in appropriate numbers.

Pluripotent stem cells possess the ability to differentiate into cells inherent to most adult tissues. Pluripotent stem cells promise to dramatically alter and extend our ability to both understand and treat many of the chronic illnesses for which there is no current management. The medical and industrial application of all stem cells for therapeutic usage requires the ability to generate large numbers in vitro. The production of monoclonal antibodies through in vitro immune systems, the production of islets for diabetes treatment, and the production of neural precursors for neural related dysfunction are just a few of the human disease areas needing a steady reliable production of specific cell types. The economic significance of this project is dramatic. The monoclonal antibody application alone is a multibillion dollar industry. The production of adequate numbers of stem cells for patient-specific or, where necessary, donor-derived, will likewise be a multi-billion dollar industry. The farming of stem-cell manufactured products secreted in cell culture will likewise be a multi-billion dollar industry and the integration of these secreted products into medically useable therapeutic application will be a multi-multi-billion dollar industry.

The National Institutes of Health estimates that the annual cost of diabetes to the United States is $132 billion.

(http://diabetes.niddk.nih.gov/dm/pubs/statistics/index.htm# 14).

In 10 years, the annual cost of Alzheimer's disease to Medicare and Medicaid in the United States alone will rise from $50 billion to more than $82 billion. The NAA is looking to increase the funding for research at the National Institutes of Health to $1 billion a year. It's currently funded at $580 million a year. .shtml)

In the USA, current statistics suggest an annual cost of $400 billion for heart treatment and lost productivity; with 900,000 heart attacks and strokes; 1.2 million angioplasties; the 500,000 bypass operations and a million hospitalizations for heart failure (www.americanheart.org)—and these statistics will only continue to rise with an aging population and the dramatic increase of diabetes and hypertension in the younger population.

With scaled-up production of such cells and their manufactured products, availability is no longer a limiting factor. Cells can then be used therapeutically for organ-appropriate infusions. Of particular benefit is the potential to generate autologous cells for each patient's usage—this can be done rapidly and effectively and negate immune rejection as the cells are perfectly (MHC/HLA) matched. Appropriate delivery routes are or will be technically established to ensure rapid delivery of stem/progenitors to target tissues.—Liver regeneration is noted following introduction of healthy liver cells to damaged hepatic tissues, e.g. as a result of Hepatitis infection or alcohol abuse. Immune disorders may be treated by administration of appropriate lymphocytic progenitors, muscle wasting by the introduction of skeletal muscle-derived satellite stem cells, diabetes through transplanting pancreatic islets to name but a few applications.

Neuronal growth and differentiation is important in a wide variety of processes from neuronal development to memory. The ability to modulate such growth and differentiation is important for the study of neurons as well as for the treatment of diseases and conditions that involve neuronal growth, degradation, or injury. For example, many neurodegenerative diseases, such as Alzheimer's disease and Huntington's Disease, are characterized by the death of neurons. Traumatic injuries to nerves, including trauma to the spine and damage caused by ischemic cerebral stroke, can involve neuronal death. Any of these conditions can potentially be treated or symptomatically alleviated temporally using agents that promote growth or prevent the loss of neurons. Additionally, the generation of long-term memory is believed to result from strengthening of neuronal connections and synaptic remodeling and stem cell infusions may be important in providing the microenvironment necessary for in situ cells to resume/re-establish normal functional cellular physiology.

Cells are only part of a potential solution to medical need. Harvest and use of the unique products manufactured by stem cells introduces an entirely new class of pharmaceutical for development and use in numerous modes of delivery products for therapeutic purpose and application. (i.e. ranging from but not limited to induction of healing with minimal scarring such as in the treatment of burn victims, healing of diabetic ulcers, microbial barriers of all types).

### Problem the Invention Addresses

Cell culture medium conducive to the support and expansion of stem cells and their products remains largely undefined. Because the stem cell micro-niche is possibly dynamic-variable yet specific to each type of stem cell due to site derivation, additives (i.e. additional enhancements to the basic culture medium) are often trial and error, subjective and used only if they show promise in sustaining or impacting specific cellular populations. These additives, although costly, may ultimately be widely adopted if they have demonstrated promise in culture promotion and sustenance. Need for culture medium conducive to the growth and maintenance of stem cells is currently driving and directing the marketing of numerous cell culture media. Media formulation is becoming a billion dollar industry based on the varied needs of stem cell researchers. As stem cells continue into prominence as the “new therapy” of the future, this need will hold researchers captive in a restrictive market.

Cell culture medium and culture supplements conducive to basic and routine sustenance must be well-defined and capable of standardization. Biological accessibility must supply, supplement, replenish and/or sustain available nutrients amenable to proliferative or other metabolic dynamics of and impacting an expanding population. The narrowly-defined physiological culture environment of neutrality (pH 7.0-7.5) necessary to support cellular metabolism must be maintained.

Ideally, a ‘universal culture medium’ should contribute to at least one of the following:
1. metabolic enabling or enhancement of some measurable parameter within the cellular population
2. enabling or enhancing availability of nutrients to cells
3. enabling or enhancing usage of nutrients by cells
4. distinct and positive effect on cellular dynamics (i.e. proliferation and increased synthesis of stem cell manufactured products such as cytokines and chemokines)
5. non-interference with normal cellular metabolics (i.e. signaling)
6. non-toxicity or increased protection from the effects of toxicity to the cellular population under culture conditions
7. provide additional benefits to the cellular population which, in absence of the ‘universal culture medium’, would be lacking

For instance, cardiac and other stem cells are difficult to grow because cytokines, chemokines and other growth factors remain undefined. Accordingly, all culture conditions in current use are less than ideal to support their growth requirements and dynamics. Moreover, stem cells may be slow to divide which may become a drawback to clinical usage. Steele et al., 2005, reported that cardiac stem cells appear to divide every 40 hours. (Steele A, Jones O Y, Gok F, Marikar Y, Steele P, Chamizo W, Scott M and Robert J. Boucek, Jr. (In Memoriam: Robert A. Good, M.D. Ph.D., D. Sc.) Stem-Like Cells Traffic From Heart Ex Vivo, Expand In Vitro, And Can Be Transplanted In Vivo. Journal Heart and Lung Transplantation. JHLT:24(11):1930-1939. 2005.

Additionally, non-passage of cultured cells, an approach which often is utilized in stem cell culturing so as not to disturb the original growth environment of the culture eventually promotes environmental buildup of metabolic by-product which becomes toxic to the cells a researcher is attempting to grow. This obviously presents a serious problem demanding an effective solution. A culture medium should possess a defined purity and associated standardization in production (quality control/assurance) and, moreover, contribute to experimental enabling while facilitating cost-efficiency via their usage.

Obviously any culture additive demonstrating enhanced capacity to meet metabolic requirements of the cellular population for basic sustenance and for proliferative demands is highly significant not only to the culturing of these cells but also for the attendant manufacture of their inherent and unique products with subsequent availability for clinical applications.

Polysaccharides and polypeptides extracted from rice have been shown to induce neuronal outgrowth. The invention includes methods of inducing neuronal outgrowth using a medium additive from extracts of rice which provides polysaccharides, as well as associated proteins or polypeptides. Cell growth can be stimulated in cells in culture or potentially in cells within an animal or patient. Growth stimulation has application to understanding and treatment of neurodegenerative diseases including, for example, Parkinson's disease, Alzheimer's disease and multiple sclerosis and conditions such as stroke, brain injury and spinal cord injury. Such compounds also can be used to increase neural stem or progenitor cell numbers in culture or in an animal, and to innervate engineered tissue.

In conclusion, stem cells can produce new cells to repair damage to any tissue in the body. Likewise, their cellular manufactured products may hold immense potential for application in both reparative and regenerative medicine. Stem cells are present in all body tissues and organs but some, like bone marrow and blood, are more accessible than others, like liver and brain. However, stem cells exist in very small numbers in marrow and blood, and need to be extracted and then increased in number (“expanded”) before they and their unique cellular products can be used clinically. Currently, many attempts are being made to accomplish the aim of providing stem cells in sufficient numbers to perform tissue-specific stem cell transplantation. Little work has been done to date to promote stem cell-specific manufactured cell products into useful medical products.

### SUMMARY OF THE INVENTION

This invention provides a novel method using processed rice for universal application as a culture medium additive for routine culturing of cells in general laboratory and research use, including stem cells and their products. This ‘universal culture’ additive provides a method to maintain various cultured cells, including delicate stem cells, in an environment which contributes to the following: metabolic enabling or enhancement of measurable parameters within the cellular population, availability and usage of nutrients to cells; distinct and positive effects on cellular dynamics (i.e. proliferation and secretion of manufactured cellular products such as cytokines); non-interference with normal cellular metabolics (i.e. signaling); non-toxicity or increased protection from toxic effects inherent to build-up of cellular metabolites/byproducts in culture conditions; and, provides additional benefits to the cellular population which, in absence of the ‘universal culture medium’, would be lacking.

This culture medium additive made from any variety of processed rice and added to combinations of other additives creates a ‘universal culture medium’ used for the support and expansion of cells including cardiac and other stem cells, their proliferation, differentiation, manufactured secretory products, anti-toxic effects and nutritive requirements. The ‘universal culture medium’ is amenable to methods enabling multi-dimensional (3-D) growth matrices, observation of cellular dynamics and analyses of homo- and heterogeneous populations for cells including but not restricted to fat, bone, peripheral muscle, smooth muscle, bone marrow/luminal components, embryonic and early embryonic and developmentally-committed cellular layers such as endo, ecto and mesoderm from which all cells types are ultimately derived, connective tissue, cardiac stem cells and all other cells including cancerous cells of all lineages) grown in medium using this culture additive.

### DESCRIPTION OF THE INVENTION

The invention provides a method with potential to maintain various cultured cells, including delicate stem cells, and their derived products, in an environment of enhanced, available cellular nutrition while providing protection from toxicity inherent to build-up of cellular metabolites in culture conditions. Accordingly, this invention has the potential to act as a ‘universal culture medium’.

Enhanced cellular nutrition is expected to directly contribute to an increase in cellular proliferation thereby enabling an up-scaling of production of cultured cells for experimental or clinical applications. Protection from toxicity enables an extended window for maintenance and expansion of difficult-to-grow or slowly dividing cells as well extending time for up-scaling of mainstream populations while offering protection from DNA damage routinely observed under long-term culture conditions. Enhanced cellular nutrition is further expected to result in an increase in amount of unique manufactured and secreted cells by the expanding population. Up-scaling of production of cultured cells by default will also lead to increased amounts of unique cellular manufactured products for culling and application and use in new medical products.

This is a novel culture medium with a form of processed rice as a base. One form is prepared using a proprietary process including “mechanical hydrolysis”, precooking, partial and pregelatinization, and/or micronization, which ensures a pure, natural product of carbohydrates, protein, vitamins and minerals. In clinical trials in humans the processed rice has been shown to have cardiovascular impact via lowering of cholesterol levels. Additionally, lowering of and stabilization of blood sugar values in diabetics has been reported. Evidence from in vitro experiments further suggests that the processed rice can act in a neuron-protective and regenerative capacity in experiments wherein neuronal cells were exposed to neurotoxic agents. Subsequent research demonstrated this processed rice to significantly increase mitochondrial function of cells even when under duress from potent neurotoxins as measured by MTT calorimeter.

Recently, a variety of homo- and heterogeneous populations for cells including but not restricted to fat, bone, peripheral muscle, smooth muscle, bone marrow/luminal components, embryonic and early embryonic and developmentally committed cellular layers such as endo, ecto and mesoderm from which all cells types are ultimately derived, connective tissue, cardiac stem cells and all other stromal cells grown in medium using this culture additive appear to evidence enhanced metabolic enabling, an increase in proliferation, enhanced ability and non-interference in signaling, and increased protection from the effects of toxicity, while providing bio-available nutrients to the cells.

### DETAILED DESCRIPTION OF THE INVENTION

### The ‘Universal Culture Medium’:

Components: Polysaccharides and polypeptides derived from a processed rice prepared using a proprietary mechanical hydrolysis and other processes to promote cellular function.

### Preparation:

1. Dissolve 0.5 g-4.0 g processed rice in 40 ml DMEM (Basic Culture medium).
2. Incubate at 37 C overnight. Vortex vigorously.
3. Autoclave.
4. Sterile-filter solution or filter/re-autoclave.
5. Add supernatant to 500 ml DMEM complete prepared as: DMEM, 50 ml 10% fetal calf serum, 6 ml penicillin-streptomycin antibiotic
6. Mix thoroughly by shaking.
7. Pre-warm to 37 C.
8. Use at 100% strength. Fill culture flasks in sterile culture hood.
9. Seed culture flasks with pre-determined number of cells (use hemocytometer).
10. Incubate at 37 C in “clean” incubator.
11. Check flasks frequently using inverted microscope to ascertain patent population.
12. All culture feedings should be performed using stock solution.
   **NB: If routine 3 day feeding schedule is maintained, culture will require increased passages due to increased proliferation dynamics resultant from the processed rice as culture additive.
All experimental manipulations may be routinely performed while employing this rapid-growth medium.

### Protocol:

Five strengths of the ‘universal culture medium’ using the processed rice additive were prepared as described:
Varying quantities of formula in 40 ml DMEM were used.
Culture flasks were prepared under sterile conditions and seeded from source cultures using equivalent numbers of cells.
Cells cultured included: fat cells, peripheral muscle cells, fractionated bone marrow cells, and cardiac stem cells.
Cultures were maintained at 37 C, 5% CO2, 95% humidity.
Cultures were checked daily using an inverted microscope.
Stock culture medium was maintained within a physiological pH of 7.4-7.5.
Cells were fed every three days.
Cells were not passaged.

### Experimental Results Supporting the Invention:

The ‘universal culture medium’ at various concentrations supported the growth of all cultured cell types tested. Under culture conditions employing the optimal quantity or amount of the ‘universal culture medium’ in complete DMEM a proliferative burst was clearly evident in all populations but markedly so in cardiac stem cell populations.

This observation is highly significant because cardiac stem cells are difficult to grow and because cytokines supporting their growth remain undefined. Accordingly, all culture conditions in current use are less than ideal to support their growth requirements and dynamics. Moreover, cardiac stem cells have been reported to divide every 40 hours—a drawback to their usage in clinical settings.

Obviously any culture medium demonstrating enhanced capacity to meet metabolic requirements for sustenance and for proliferative energy demands is highly significant not only to the culturing of these cells, but also to their availability for clinical applications.

Non-passage of cultured cells as often used in stem cell culturing with passage defined with addition of fresh media can promote environmental build up of metabolic by-product which is toxic to cells. However, cultures appeared to remain viable and proliferative. No significant increase in cell deaths was observed. This observation is highly significant. Significant also was a marked increase in maintenance of a medium pH of 7.5 throughout the experiment. The preceding suggests that toxins were slow to build even in the face of increased proliferation and that the cells were protected from associated effects of toxic byproducts of metabolism which obviously were inherent to the culture dynamics. Additionally, the preceding environment enables concentration and maintenance of manufactured and secreted cellular products such as stem cell specific cytokines, chemokines and growth factors for use in additional applications.

It will be readily apparent to those skilled in the art of laboratory science and stem cell research, that various modifications and changes can be made to the described ‘universal culture medium’ without departing from the spirit and scope of this invention. Accordingly, all such modifications and changes as fall within the scope of the appended claims are intended to be included in this invention.

What is claimed is:

1. A universal culture medium additive made from processed rice, rice extract, fermented rice or rice nutrients and added to combinations of additives and/or culture media to create a ‘universal culture medium’ to support nutrition, proliferation, expansion, differentiation, and maintenance dynamics inherent to all plant and animal (including human) somatic cells including, somatic stem and germline cells of all natural and engineered derivation and creation and similarly, all biological microorganisms such as bacteria, virus and fungi, including their unique cellular manufactured and secreted products, respectively.

2. The method in accordance with any of the claim 1 wherein the following types of rice are chosen for the universal culture medium additive: white rice or sticky (glutinous) rice, brown rice, basmati rice, red or black rice.

3. The method in accordance with claim 1 where ‘universal culture medium’ is prepared by:
(a) dissolving 0.1 g-4.0 g of processed rice culture additive in 40 ml DMEM (Basic Culture Medium),
(b) which is then incubated at 37 C overnight
(c) after which it is put through vigorous vortex and autoclaved
(d) then put through a Sterile-filter or filter/re-autoclave process
(e) to which supernatant is added to 500 ml DMEM, Dulbecco/Vogt modified Eagle's minimal essential medium, (DMEM being a basic medium that is purchased commercially and modified by each researcher to suit growth requirements with cytokines, growth factors, and cells, and is a basic fluid that has to be modified to keep specific cells nourished, alive and metabolically active, containing vitamins, amino acids, iron and glucose that is used as a basic starting medium for most types of cells—such as human, monkey, hamster, mouse, rat, chicken—complete prepared in any amount)
(f) with proportions best maintained at DMEM 500 ml; 50 ml FCS (NOTE: FCS may be proportioned to 20% or 100 mls as required by cell line, and the solution mixed thoroughly by shaking and then pre-warmed to 37 C).

4. The method in accordance with claim 1, where ‘universal culture medium’:
(a) is used at 100% strength
(b) with culture flasks filled in a sterile culture hood
(c) then culture flasks are seeded with pre-determined number of cells using a hemocytometer or other method of cell counting
(d) and then incubated at 37 C in “clean” (sterile), humidified incubator
(e) after which the flasks are frequently checked using inverted microscope to ascertain patent population
(f) with all culture feedings being performed using stock solution
(g) and a routine 3 day feeding schedule is maintained
(h) with culture eventually requiring increased passages due to increased proliferation dynamics.

5. The method in accordance with claim 1 where all experimental manipulations may be routinely performed while employing this rapid-growth medium.

6. The method in accordance with claim 1, where five strengths of the universal culture medium are prepared as described:
(a) using varying quantities of formula in 40 ml DMEM/additive mixtures
(b) with the culture flasks prepared under sterile conditions and seeded from source cultures using equivalent numbers of cells
(c) with the cells cultured to include: fat cells, bone cells, peripheral muscle cells, fractionated and unfractionated bone marrow cells, fibroblasts, endothelial, stromal components, embryonic tissue layers and/or cardiac stem cells
(d) the cultures are then maintained at 37 C, 5% CO2, 95% humidity
(e) after which the cultures are checked daily using an inverted microscope
(f) and the stock culture medium maintained within a physiological pH of 7.4-7.5
(g) with the cells being fed every three days and are not passaged.

7. The method in accordance with claim 1 supported the growth of all cultured cell types tested, with concentrations of 0.5 g-3.0 g effectively supporting increased but moderate cellular proliferation proportional to concentration of culture medium, and all cultured cell types evidenced sustained proliferation and culture maintenance of pH without passage, an indication of slow toxic buildup and cytoprotection from toxic byproducts of metabolism, under culture conditions employing a higher concentration of universal culture medium in complete DMEM/additive, with a proliferative burst clearly evident in all populations tested.

8. The method in accordance with claim 1, wherein the ‘universal culture medium’ is produced from a rice extract consisting of polysaccharides and polypeptides that have been manufactured through the following process:
1. mechanical hydrolysis using heat of between 80-140 degree Celsius (176-284. deg. Fahrenheit)
2. total time of the entire process less than 30 minutes
3. moisture of the end-product <10%
4. particle size of the end-product <1,000 microns
5. end-product containing polysaccharide 60-98% by weight
6. end-product containing polypeptide 2-40% by weight

9. The method in accordance with claim 1, wherein said ‘universal culture medium’ may be formulated with or be comprised of any of, but not limited to, the following substances: serum or serum-substitute/replacement, serum (Fetal Bovine serum—FBS-DMEM) or non-serum medium, hormones (insulin and other hormones), growth factors (GGF, NDF, TGF-beta, Mitogen), growth enhancers, steroids, enzymes (collagenase), vitamins (ascorbic acid, B-vitamins), minerals (calcium, magnesium, zinc, potassium, sodium), trace elements, proteins (NM23 protein, laminin, fibronectin, vitronectin, lectins, collagen, gelatin, amino acids (cysteine or SPARC), carbohydrates, proteoglycans, antioxidants, fatty acids, ligands (thrombopoetin), chondroitin sulfate, food substances, concentrates or extracts, resovist RTM (iron), antibiotics, synthetic drugs or compounds, algae or seaweed, botanical herb, chemicals, preservatives, chemokines, methyl cellulose, bioactive compounds, bioflavonoids, buffers, suppressors, stimulants, stimulators, various alcohols, amniotic membrane, antibodies, polymers, organic compounds, interleukins, mammalian or human glands or organs, DMSO or MSM, heparin sulfate, metals (colloidal silver), biomaterial shell, vectors (viral and retroviral), cytokines (IL-3, LIF), oncostatin M, forskilin, Dulbeccos Modified Eagle Medium, including culture additives or conventional/commonly used culture or cell medium and any combination of the above substances listed and at variable concentrations.

10. The method in accordance with claim 1, wherein said ‘universal culture medium’ can increase proliferation, manufacture and secretion of unique products of cellular metabolism, provide anti-toxic effects and enable multi-dimensional observation of a variety of cells and stem cells (fat cells, bone cells, peripheral muscle cells, fractionated and unfractionated bone marrow cells, fibroblasts, endothelial, stromal components, embryonic tissue layers and cardiac stem cells, and all other cells including cancerous cells and lineages) grown in medium using this ‘universal culture medium’.

11. The method in accordance with claim 1 wherein the ‘universal culture medium’ is developed for the culturing of multipotent, pluripotent and totipotent stem cells (mammalian including human embryonic) of all kind, adult stem/progenitor cells from all organs and committed, differentiated cells lineages such as neuronal, lymphatic, leukocytic, osteocytic, chondrocytic, hepatic, enteric, stromal, mesenchymal vascular, myogenic, and all other endo, meso and ectodermal differentiated derivatives including their manufactured and secreted cellular products and additionally any cancerous transformations and derivatives of the preceding.

12. The method of claim 1 wherein the development of said “universal culture medium” creates a culture medium that can act in accordance with a true 3-D matrix promoting and sustaining growth of stem cell multi-cellular “spheroids” within the matrix as opposed to synthetic or constructed adherent surfaces enables cellular-driven formulations having uniform access, such as to nutrients and metabolite diffusion, but most importantly enables mimicry of the in vivo 3-Dimensional physiological functional organismal cytoarchetecture or alternatively supports an in-vitro proliferative unit mimicking or representative of various stages of cellular proliferation and differentiation within the organ “proper” or alternatively is used in this capacity to maintain spheroids to further increase seeding/re-seeding of cultures and differentiation of cellular populations or to isolate or promote a unique spheroid-inherent cellular manufactured product.

13. The method in accordance with claim 1 wherein the biological accessibility of the culture additive supplies, supplements, replenishes and/or sustains available nutrients amenable to proliferative and metabolic dynamics of and impacting an expanding population of cells and stem cells.

14. The method in accordance with claim 1 wherein the ‘universal culture medium’ provides and maintains a narrowly-defined physiological culture environment of neutrality (pH 7.0-7.5) necessary to support, or consistent with all aspects of, cellular metabolism.

15. The method in accordance with claim 1 wherein said ‘universal culture medium’ is conducive to basic and routine sustenance of a somatic cell lineage or stem- or stem/progenitor cell lineages which are currently or will be concurrent with biological understanding and will become well-characterized molecularly and capable of standardization with concordant technology.

16. The method in accordance with claim 1 wherein said ‘universal culture medium’ provides and is conducive to metabolic enabling or enhancement of some measurable parameter within the cellular population.

17. The method in accordance with claim 1 wherein said ‘universal culture medium’ provides a means of driving the cells proliferatively while they are being highly nourished while concurrently maintaining a controlled microenvironment—an aspect which has proved to be scientifically challenging.

18. The method in accordance with claim 1 wherein said ‘universal culture medium’ enables or enhances availability and usage of nutrients to cells.

19. The method in accordance with claim 1 wherein said ‘universal culture medium’ creates distinct and positive effects on cellular dynamics (i.e. proliferation and the attendant secretion of manufactured cellular products which may hold inherent biological common and/or uniqueness for that cell population and may be found to hold similar common and/or unique properties for use and application in biomedicine and medical application).

20. The method in accordance with claim 1 wherein said ‘universal culture medium’ provides non-interference with and enhancement of normal cellular metabolics (i.e. signaling).

21. The method in accordance with claim 1 wherein said ‘universal culture medium’ provides a non-toxic environment and/or increased protection from the effects of toxicity to the cellular population under culture conditions.

22. The method in accordance with claim 1 wherein said ‘universal culture medium’ incorporates or utilizes perfusion, incubation, washing, thawing, aspiration, cryopreservation, suspension, separating (including fluorescence, magnetic beads), stimulants for differentiation (i.e. LY294002, nicotinimide, azacytidine, oxytocin), toll-like receptors, transference (transfection, infection, microinjection, cell fusion, protoplast fusion or ballistic bombardment including labeling using nanoparticles/nanocrystals/magnetic particles, agitation, electroporation and any combination or similar process thereof or new process which will become in routine use for stem cell research.

23. The method in accordance with claim 1 wherein ‘universal culture medium’ processes are carried out at varying time limits from one second to an unspecified and unlimited amount of time.

24. The method in accordance with claim 1 wherein said ‘universal culture medium’ is used in an intracellular and extracellular liquid, porous or solid culture matrix or used without this matrix.

25. The method in accordance with claim 1 wherein said ‘universal culture medium’ is prepared in a tissue culture substrate (tissue culture flask, single or multi-wall plate or chamber or rotary culture apparatus).

26. The method in accordance with claim 1 wherein said ‘universal culture medium’ is used with or without feeder layers.

27. A method in accordance with claim 1 of culturing human embryonic stem cells comprising: culturing the stem cells in a culture including or alternatively essentially free of mammalian fetal serum and in a stem cell culture medium with the universal culture medium inclusive or not inclusive of amino acids, vitamins, salts, minerals, transferrin or a transferrin substitute, insulin or an insulin substitute, albumin, and a fibroblast or other cell-derived growth factor that is supplied from a source other than just a feeder layer or a synthetic or partially synthetic serum substitute of any composition.

28. A method in accordance with claim 1 of culturing human embryonic stem cells that are: (i) capable of indefinite proliferation in vitro in an undifferentiated state; (ii) capable of differentiation to derivatives of all three embryonic germ layers (endoderm, mesoderm, and ectoderm) even after prolonged culture; and (iii) maintain a normal karyotype (are euploid) throughout prolonged culture, with these cells therefore referred to as being pluripotent.

29. The method in accordance with claim 1 wherein said ‘universal culture medium’ is used in either positive selection (isolation of cells) or negative selection (removal of cells).

30. The method in accordance with claim 1 wherein said ‘universal culture medium’ is used with or without blocking agents (gelatin, sucrose, trehalose).

31. The method in accordance with claim 1 wherein said ‘universal culture medium’ is used with or without differentiation stimulators or suppressors.

32. The method in accordance with claim 1 wherein said ‘universal culture medium’ is a sterilized dried substrate in moisture-resistant packages.

33. The method in accordance with claim 1 wherein said ‘universal culture medium’ is used with variable oxygen conditions.

34. The method in accordance with claim 1 wherein said ‘universal culture medium’ is used with or without reagents for binding to, associating with or recognizing an antigen (monoclonal or polyclonal antibodies, ligands, or proteins).

35. The method in accordance with claim 1 wherein said ‘universal culture medium’ is used with or without inducing agents.

36. The method in accordance with claim 1 wherein said ‘universal culture medium’ is used in ex vivo or in vitro.

37. The method in accordance with claim 1 wherein said ‘universal culture medium’ is used for the cultivation of cells or stem cells on microcarriers in vessels.

38. The method in accordance with claim 1 wherein said ‘universal culture medium’ is used with or without surfactants such as glycerine, phosphatydal choline, polysorbate 80.

39. The method wherein said ‘universal culture medium’ is used in accordance with claim 1 with stem cells from any human or other mammalian, non-mammalian or alternatively plant sources including but not restricted to: embryonic germ layers and developing organs, umbilical cord blood, circulating (peripheral) blood, bone marrow, hair follicle, tissue, all organs, connective tissue components, structures and glands or their equivalents.

40. The method wherein said ‘universal culture medium’ is used in accordance with claim 1 for the production of all types of stem cells to include: cardiac, embryonic, vascular, bone, organ-specific (somatic) including digestive, glands, connective tissue, nerve or to maintain their related production, maintenance and processing in experimental set-ups of all types useful for the production of these cells.

41. The method wherein said ‘universal culture medium’ is used in accordance with claim 1 to carry a human, chick, mouse, rat, pig, monkey or alternatively, fish embryos (such as zebra fish) in it to a beating heart stage.

42. The method wherein said ‘universal culture medium’ is used in accordance with claim 1 to support the initiation of growth and completion of various developmental stages of growth in undeveloped embryos derived from natural fertilization, in vitro fertilization, nuclear transfer into eggs, nuclear transfer in to somatic cells, clonal derivations and genetic manipulations to create or invoke stem cell status on somatic cells, and the co-incident signaling in each case which goes on to sustain these cellular populations during growth, differentiation and application.

43. The method wherein the said ‘universal culture medium’ and all derived or biologically-created or manipulated stem cells and their associated manufactured and derived products developed from such, as in claim 1, is used for the benefit of degenerative/disabling/progressive disease or disease associated processes as evidenced in but not restricted to: (Alzheimer, Multiple Sclerosis, ALS, Paralysis, Parkinson's and other Neurological Diseases, Diabetes, Cardiac, Liver, Kidney, Skin, Bone, Optical, Lung), Sexual and Reproductive Glandular and Organ Dysfunction, and all Glandular and Vital Organs, as well as for reversal of disease processes through Cellular Re-Creation, Organo-Re-Scaffolding, other approaches to Artificial Creation, and Cellular Repair and Regeneration and Reactivation either in vivo, in vitro or ex-vivo or de novo of failing or failed vital or non-vital organs including but not restricted to heart, brain, hematopoietic organs, spinal cord, peripheral nervous system, visual system including eye.

44. The method wherein said ‘universal culture medium’ and the stem cells and their cellular manufactured, unique and non-unique secreted products developed from such, as in claim 1, is used and extended to diverse applications such as Cosmetic Therapies including but not restricted to Regeneration and Revitalization.

45. The method wherein said ‘universal culture medium’ and the stem cells and their products developed from such, as in claim 1, is used as a preventive and/or therapeutic agent wherein the disease which accompanies tissue injury is any one of but not restricted to the neural disease, respiratory organ system disease, cardiovascular disorders, hepatic disease, pancreatic disease, digestive organ system disease, renal disease and skin disease.

46. The method wherein the said ‘universal culture medium’ and all organ-derived cells, including stem cells and progenitor cells and their products developed from such, as in claim 1, is used as a preventive and/or therapeutic agent wherein the disease, disorder or other dysfunction may arise from or be based in any tissue or organ within the body and stated cell sources are used for any reparative or regenerative approach to healing.

47. The method wherein the said ‘universal culture medium’ and the stem cells and their products developed from such, as in claim 1, is used as a palliative or a solution for skin and tissue injuries, wound care, burns, and post-surgical incision care.
